(19) United States
(12) Patent Application Publication
(10) Pub. No.: US 2020/0276389 A1
(43) Pub. Date: Sep. 3, 2020

(54) A Container for at least a First Injectable Medicament and Injection Device

(71) Applicant: SANOFI, Paris (FR)

(72) Inventor: Thomas Klemm, Frankfurt am Main (DE)

(21) Appl. No.: 16/765,234

(22) PCT Filed: Nov. 19, 2018
(86) PCT No.: PCT/EP2018/081705

§ 371 Date: May 19, 2020

(30) Foreign Application Priority Data
Nov. 21, 2017 (EP) 17306610.1

(51) Int. Cl. A61M 5/19; A61M 5/24; A61M 5/315

(52) U.S. Cl. CPC A61M 5/19; A61M 5/31596; A61M 5/2425; A61M 5/2459

(57) ABSTRACT

The present disclosure relates to a container (10) for at least a first injectable medicament (60), the container comprising:—an elongated body (11) having a tubular-shaped sidewall (12) extending along a longitudinal axis (z) and having a distal end (13), wherein the elongated body (11) comprises a hollow space (15) with a needle receiving subsection (17) adjacent to the distal end (13),—an outlet (40) at the distal end (13) of the body (11),—a piston (20) arranged inside the elongated body (11), sealingly engaged with the sidewall (12) and slidable along the longitudinal axis (z) relative to the sidewall (12),—a membrane (30) arranged inside the elongated body (11) sealingly engaged with the sidewall (12) and being at least one of slidable and deformable along the longitudinal axis (z) relative to the sidewall (12),—a first cavity (16) confined by the sidewall (12), the outlet (40) and the membrane (30), wherein the first injectable medicament (60) is located in the first cavity (16),—a second cavity (18) confined by the sidewall (12), the piston (20) and the membrane (30), wherein the piston (20) and the membrane (30) are separated from each other by a distance along the longitudinal axis (z),—wherein the outlet (40) is configured to receive or to hold a needle (50) extending from the distal end (13) into the needle receiving subsection (17), and—wherein in an initial configuration, the membrane (30) is located outside the needle receiving subsection (17) and wherein the membrane (30) is displaceable or deformable relative to the sidewall (12) to such an extent that at least a portion (31) of the membrane (30) enters the needle receiving subsection (17).

### TECHNICAL FIELD

The present disclosure relates to the field of injection devices and in particular to containers containing at least a first injectable medicament. The disclosure also relates to injection devices and to containers containing at least a first and a second injectable medicament that are to be injected sequentially, i.e. one after the other.

### BACKGROUND

Certain disease states require treatment using one or more different medicaments. Some drug compounds need to be delivered in a specific relationship with each other in order to deliver the optimum therapeutic dose.

There are a number of potential problems when delivering two active medicaments or “agents” simultaneously. The two active agents may interact with each other during the long-term, shelf life storage of the drug formulation. Therefore, it is advantageous to store the active components separately and combine them at the point of delivery, e.g. injection, needle-less injection, pumps, or inhalation. Prior to or during injection the active components must be mixed appropriately.

Additional problems could arise where a multi-drug compound therapy is required, because many users cannot cope with having to use more than one drug delivery system or make the necessary accurate calculation of the required dose combination. This is especially true for users with dexterity or computational difficulties. In some circumstances it is also necessary to perform a priming procedure of the device and/or of a needle or cannula before dispensing the medicaments. Likewise, in some situations, it may be necessary to bypass one drug compound and to dispense only a single medicament from a separate reservoir. Further, for some drug combinations for which this delivery of two medicaments in a single injection step is desirable, it may be additionally desirable for the two medicaments to be delivered sequentially (i.e. one after the other, with minimal or no opportunity for mixing).

Avoidance of mixing of the two drug formulations might have several advantages. For example, it is known that the pharmacokinetics of certain drugs is critically dependent on their concentration. By delivering two drugs sequentially with no mixing the optimal concentration of each drug for optimal pharmacokinetics can be maintained. In addition, certain drugs have to be formulated in particular solvent environments (e.g., a specific pH range) to remain in solution. By delivering two drugs sequentially with no mixing the optimal pH range and therefore solubility can be maintained during delivery.

Accordingly, there exists a need to provide a device for a short-term or long-term storage of at least two liquid medicaments. It is further desirable to provide and to enable a sequential delivery of two or more medicaments with a single injection or with a delivery step that is simple for the user to perform.

### SUMMARY

In one aspect there is provided a container for at least a first injectable medicament. The container comprises an elongated body having a tubular-shaped sidewall extending along a longitudinal axis (z) and having a distal end. The elongated body comprises a hollow space with a needle receiving subsection adjacent to the distal end. The container further comprises an outlet at the distal end of the body. Via the outlet the at least first injectable medicament can be expelled from the hollow space of the elongated body.

The container further comprises a piston arranged inside the elongated body. The piston is sealingly engaged with the sidewall of the body and is slidable along the longitudinal axis relative to the sidewall. By longitudinally or axially displacing the piston relative to the sidewall, hence relative to the body, the at least first injectable medicament contained inside the body can be expelled via the outlet. The piston may comprise a bung or a stopper, e.g. in form of a rubber bung or rubber stopper.

The container further comprises a membrane arranged inside the elongated body. The membrane is sealingly engaged with the sidewall. The membrane is at least one of slidable and deformable along the longitudinal axis relative to the sidewall. Both, the piston and the membrane serve to divide the hollow space of the elongated body into at least one or several separated compartments, i.e. at least into a first cavity and a second cavity. The membrane might be sealingly and slidably engaged with the sidewall of the body. When slidably engaged with the sidewall of the body the membrane might be substantially inflexible.

With other examples the membrane can remain stationary with the sidewall and hence with the body of the container, at least with an outer circumference being in contact and/or in permanent mechanical engagement with the sidewall of the container. With such examples the membrane might be elastically deformable with regard to the longitudinal axis. Here, only a portion, typically a central portion of the membrane is deformable with regard to the longitudinal axis and is hence displaceable relative to the longitudinal axis compared to a circumferential part of the membrane remaining in positional engagement with the sidewall of the body. The membrane may be also both, slidable and deformable along the longitudinal axis relative to the sidewall.

The container further comprises a first cavity confined by the sidewall, the outlet and the membrane. The first injectable medicament is located in the first cavity. Typically, the first cavity is filled with the first injectable medicament. The first injectable medicament may be contained in the first cavity in liquid form. The first cavity may be prefilled with the liquid first injectable medicament. Typically, the first cavity is located at or near a distal section of the elongated body. Towards the distal end the first cavity may be closed or confined by the outlet. Towards an opposite end, hence towards the proximal end, the first cavity may be confined and closed or sealed by the membrane. A distal seal of the first cavity may be provided by the outlet. A proximal seal of the first cavity may be provided by the membrane.

The container further comprises a second cavity that is confined by the sidewall, the piston and the membrane. Here, the piston may form a proximal seal for the second cavity whereas the membrane forms a distal seal for the second cavity. The first and the second cavities are divided by the membrane. By means of the membrane the second cavity is sealed against the first cavity and vice versa.

Typically and in an initial configuration the piston and the membrane can be separated from each other by a distance along the longitudinal axis. The distance between the piston and the membrane and the cross-section of the sidewall define the volume of the second cavity. Likewise, the distance between the membrane and the seal and the cross-section of the sidewall located therebetween define the interior volume of the first cavity.

The outlet of the container is further configured to receive or to hold a needle extending from the distal end into the needle receiving subsection of the elongated body. Typically, the needle may reach through or penetrate the outlet of the container. The outlet is configured to hold the needle so that a proximal end of the needle, typically in form of a tipped proximal end, is located inside the hollow space of the container and inside the needle receiving subsection. The size of the needle receiving subsection, in particular the longitudinal elongation of the needle receiving subsection is determined by the depth or length the proximal end of the needle protrudes in longitudinal direction into the interior and the hollow space of the container. Since the first cavity is confined by the outlet, the needle, when attached to the container extends into the first cavity. The size of the needle receiving subsection may vary and depends on the specific position and extension of the needle extending into the interior of the elongated body. Typically, a proximal end of the needle coincides with a proximal end of the needle receiving subsection.

In an initial configuration of the container the membrane is located outside the needle receiving subsection. Typically, the membrane is located at a proximal distance from a proximal end of the needle receiving subsection. Moreover the membrane is displaceable or deformable relative to the sidewall to such an extent that at least a portion of the membrane enters the needle receiving subsection. Typically, the membrane is displaceable or deformable in distal direction, hence towards the outlet to such an extent that at least a portion of the membrane enters and arrives at the needle receiving subsection where the respective portion of the membrane can be punctured, pierced or penetrated by the needle.

By means of the membrane and the piston two cavities can be provided, e.g. for two different liquid and injectable medicaments. The two cavities are separated by the membrane. In an initial configuration only and exclusively the first cavity is in fluid communication with the outlet. Typically, the first cavity is in fluid communication with the exterior of the container. When configured as a double-tipped an injection needle a distal end of the needle may be located inside biological tissue pierced by the needle.

As the piston is subject to a distally directed displacement, e.g. by means of a plunger or by means of a drive mechanism of an injection device a fluid pressure or gas pressure inside the second cavity will build up until the pressure in the second cavity is larger than a force or pressure required to deform or to displace the membrane in distal direction. The displacement or deformation of the membrane towards the distal direction leads to a respective build-up of a fluid pressure inside the first cavity, thereby expelling an amount of the medicament through the outlet. When the membrane is subject to a distally directed displacement relative to the body the size of the first cavity will constantly decrease, thereby expelling the at least first injectable medicament through the outlet, e.g. through the needle that is attached to the outlet.

Once the membrane has been displaced in distal direction to such an extent that it reaches the needle receiving subsection the membrane is pierced by the proximal end of the needle. At least during a dispensing operation the needle is fixed relative to the housing. As soon as the membrane has been penetrated by the needle the needle gets in fluid communication with the second cavity. Driving the piston further in distal direction then leads to a subsequent dispensing and expelling of a second injectable medicament that is located inside the second cavity. As soon as the membrane is pierced by the needle a fluid communication between a distal end of the needle and the second cavity is established. Driving of the membrane further in distal direction then leads to expelling of the medicament or of the liquid substance contained inside the second cavity.

From an operator's point of view only the piston has to be driven and displaced in distal direction. At a first phase of displacement the piston and the membrane might be displaced in unison in distal direction until the membrane is pierced by the needle. Thereafter, as soon as the membrane has been pierced or intersected by the needle, in a second phase of displacement a distally directed displacement of the piston leads to an expelling of the second medicament through the needle.

With the present container at least two injectable medicaments stored separately in the first cavity and in the second cavity, respectively, can be expelled and administered during and by a single dose dispensing procedure that is to be conducted only by displacing the piston from a proximal initial position towards a distal end position.

According to a further example the membrane is pierceable and penetrable by the needle. The membrane may comprise a sealing disc extending all over the inner cross-section of the elongated body of the container. The container and hence its tubular-shaped sidewall is typically free of any recesses. The elongated body comprises a tubular and cylindrically-shaped sidewall that is void of any recesses or protrusions. This allows for a smooth displacement of the membrane towards and into the needle receiving subsection. Along an entire path the membrane is displaced towards the outlet the membrane may remain in fluid-tight and sealing engagement with the sidewall of the body. In this way an intermixing of the first and second injectable medicaments contained in the first and second cavities can be effectively prevented. for mixing of the first and second injectable medicament may only take place after having been expelled from the outlet. On the one hand the membrane is pierceable and penetrable as well as intersectable by the needle. On the other hand the membrane provides a liquid-proof seal between the first cavity and the second cavity.

According to a further example at least a second injectable medicament is located in the second cavity. Here, the second cavity may be filled with the second injectable medicament. Access to the second injectable medicament is provided only and after the pierceable membrane has been pierced or intersected by the needle in the course of a combined distally directed displacement of the piston and the membrane or in the course of a distally directed displacement of the piston accompanied by a deformation of the membrane towards the needle receiving subsection.

The first injectable medicament and the second injectable medicament may be prefilled in liquid form in the first and the second cavities of the container, respectively. In this way the container can provide a long-term shelf life and a long-term storage of the first and the second injectable medicaments without a substantial mixing of the first and the second injectable medicaments. A combined drug formulation may be prefilled in the container and the container may be handed out to patients requiring the prescribed combination of the first and second injectable medicaments. Here, the patients themselves do not have to take care about the sequence the first and the second injectable medicaments have to be injected and/or about the quantity and/or the ratio of the first and second injectable medicaments that would be required for a prescribed injection procedure.

According to another example the membrane is substantially inflexible. A substantially inflexible membrane is particularly configured for a longitudinal displacement inside the elongated body of the container when subject to a distally directed pressure. Typically, the second cavity is filled with the second injectable medicament. Applying distally directed pressure onto the piston leads to a respective pressure build up inside the second cavity. When the second cavity is entirely or completely filled by the second injectable medicament the pressure applied via the piston is spatially homogeneously distributed thereby leading to a respective distally directed displacement of the membrane.

A substantially inflexible membrane might be rather easy to puncture by the needle. Moreover, a substantially inflexible membrane may provide a kind of a haptic feedback when the membrane gets in abutment with the needle. At that point during an injection procedure at least a slightly increased dispensing pressure may have to be applied to the piston in order to intersect or to pierce the membrane by the needle. During the entire dispensing procedure the needle is fixed relative to the body. It may be fixed or may be attached releasably or ireleasably to the distal end of the body.

A rather inflexible membrane may be further of benefit to obtain a rather smooth distally directed displacement of the membrane in response to a rising pressure inside the second cavity.

According to a further example the membrane comprises at least one or a combination of the following materials: a thermoplastic polymer, a thermoplastic elastomer, a thermoplastic vulcanizate, polyethylene (PE), polyvinyl chloride (PVC), polytetrafluoro-ethylene (PTFE), silicone rubber, halogenated butyl rubber, chlorobutyl rubber, bromobutyl rubber, chemical vapor deposited poly(p-xylylene) polymers, parylene, cork. The membrane may comprise a combination of these materials. The membrane may comprise one or several layers of the above mentioned materials in order to provide a sufficient liquid-tight seal with regard to the sidewall and in order to enable a smooth displacement of the membrane in longitudinal direction as well as to provide and to enable a well-defined piercing of the membrane by the needle.

The membrane can be made of a plastic material, such as polyethylene. Alternatively, it may comprise or may consist of a comparatively thin layer of a natural or synthetic rubber. Typically, the membrane is made of a material or a material mixture that is biocompatible. It is inert to the first and the second injectable medicament. The inside facing section of the tubular-shaped sidewall may be coated or provided with a silicone layer. The material or material mixture of the membrane and/or of the piston is typically selected for not adversely affecting the siliconization or sliding behavior of the piston or membrane relative to the sidewall of the elongated body of the container.

The membrane and the material selected for deformation of the membrane does not have to provide a total or 100%-complete diffusion barrier from a pharmaceutical point of view. The first and second medicaments are injected in the same tissue anyway. The material or material mixture the membrane is made of is selected in accordance to the combination of first and second medicaments. Typically, the membrane provides a diffusion barrier that guarantees less than 1 wt % of mixing of the pharmaceutically active ingredients of the first and the second injectable medicaments over a duration of at least one year at a temperature of about 3° C.

According to a further example the membrane comprises a thickness in a range from 10 μm to 2 mm. Alternatively and according to another example the membrane comprises a thickness from 10 μm to 100 μm. In further examples the membrane comprises a thickness ranging from 100 μm to 2 mm. In other examples the membrane comprises a thickness in a range of 100 μm to 1 mm. The membrane is comparatively thin compared to the piston. The longitudinal thickness of the piston may be at least 10 times larger, at least 20 times larger or at least 50 times larger than the longitudinal thickness of the membrane.

The piston may comprise a thickness as seen in longitudinal direction of at least 2 mm, at least 3 mm or at least 5 mm. Typically, the piston comprises or is made of a thermoplastic elastomer, such as a silicone rubber or halogenated butyl rubber. With other examples the piston comprises or is made of a cyclic olefin polymer (COP) and/or cyclic olefin copolymer (COC).

The comparatively thin membrane provides a diffusion barrier and serves as a displaceable or deformable seal between the first and the second cavities. During a dispensing action the membrane floats with the first and second injectable medicaments. The thickness of the membrane is selected in accordance to the selection of the at least first and second injectable medicaments. The density and the barrier properties of the membrane are selected in accordance to or with regard to at least one of the viscosity of active ingredients or the diffusion behavior and mixing behavior of at least one of the first and second injectable medicaments.

The membrane may comprise or may be made of a thermoplastic polymer, such as polyethylene, polyvinyl chloride and/or polytetrafluoro-ethylene. In other examples the membrane may also comprise a kind of a rubber stopper. Here it may comprise or may consist of a thermoplastic elastomer, such as silicone rubber, halogenated butyl rubber or mixtures thereof.

The membrane may also comprise a chemical vapor deposited polymer, such as parylene. The membrane may be manufactured by so called plastic vapor deposition. The membrane may be formed on a liquid surface of one of the first and the second injectable medicaments after the respective liquid medicament has been filled into the hollow space of the container. The membrane may comprise parylene N, a polymer manufactured from the p-xylylene intermediate of parylene. The p-xylylene intermediate polymerizes when physisorbed on a surface, such as on the liquid surface of one of the first and the second medicaments.

Parylene has excellent barrier properties. Formation of the membrane in situ inside the container and on top of a liquid surface of one of the injectable medicaments might be beneficial in terms of a mass manufacturing of the container. Even though the parylene or parylene N is polymerized from a high temperature state on the surface of the liquid substance the ingress of thermal energy into the injectable medicament is almost neglectable. On the one hand, the coating to be formed on the liquid surface of the injectable medicament might be comparatively thin, e.g. in a range of only a few micrometers. Moreover, since the injectable medicament may be pre-cooled and may be provided at a temperature of only 3° C.-5° C. there will be a rapid condensation of the polymer on the liquid surface. The thermal energy or heat of the chemical vapor deposition process cannot damage or harm the injectable medicament. Typically, the heat capacity of the liquid medicament is at least two to three times larger than the heat capacity of the vapor deposited polymer. Taking further into account the comparatively low mass of material required to form a membrane of a thickness in the range of only a few micrometers the in-situ coating process may only lead to a heating of the liquid medicament by less than 0.5° C. or less than 1° C.

Moreover, the condensation or polymerization of the polymers on the liquid surface leads to a condensation avalanche. As a consequence, a membrane forms as a skin on a liquid surface.

The thickness of the membrane may be governed and determined by the viscosities of the first and the second injectable medicaments or fluids contained in the first and second cavities as well as by the penetration force generally required to urge the respective medicament through the needle. Fluids or medicaments comprising a comparatively low viscosity might be separated by a comparatively thin membrane. For low viscosity fluids, e.g. for fluids that comprise a viscosity of less than 1.5 mPa s a membrane of a thickness between 10 μm to 100 μm will be sufficient. Such rather thin membranes are typically made of or comprise a thermoplastic polymer, such as polyethylene, polyvinyl chloride or polytetrafluoro-ethylene or combinations thereof.

Such membranes may be comparatively inflexible and may be easily pierced or punctured by the needle when driven in distal direction. The material or material mixture for the membrane should be selected so as to become pierced without substantive deflection or bulging. For fluids having a viscosity of larger than 1.5 mPa s the membrane may comprise a thickness ranging from 100 μm to 2 mm or from 100 μm to 1 mm. When the membrane comprises a thermoplastic elastomer, such as silicone rubber or the like it may comprise a thickness between 1 mm to 2 mm.

According to another example the membrane is substantially impenetrable to liquids. At least the membrane should be substantially impenetrable to the active pharmaceutical ingredients or agents of the at least first and/or second injectable medicaments.

According to a further example the at least first injectable medicament comprises a narcotic active ingredient or a narcotic agent. The first injectable medicament will be expelled first and before the at least second injectable medicament will be expelled through the needle. In this way the container provides sequential expelling of a narcotic agent or narcotic medicament that is followed by some other type of injectable medicament. In this way, the biological tissue being subject to an injection procedure can be narcotized by the first injectable medicament. Thereafter, the second injectable medicament can be injected. In a further example the at least first injectable medicament comprises an activator configured to activate the second injectable medicament. Mixing of the first injectable medicament and the second injectable medicament takes place in the tissue after being injected into the tissue. Vaccines or selected chemotherapeutic agents have to be activated by means of an activator. For such combinations of medicaments the activator may be provided in the first cavity of the container and the vaccine or the chemotherapeutic agent may be provided in the second cavity.

In other examples only the first cavity is provided and/or filled with the first injectable medicament whereas the second cavity is either filled with a physiologically inert substance or wherein the second cavity is even void of a liquid substance. In such a configuration the container can be used for conducting a biopsy procedure. Here, the first cavity may be filled with a narcotic agent to be injected into a portion of a selected tissue. Once the content of the first cavity has been expelled through the needle and the membrane has been pierced by the needle a fluid communication is established between the needle and the second cavity. It is then even conceivable to displace or to withdraw the piston in the proximal direction, i.e. towards the proximal end thereby generating a negative pressure in the second cavity in order to extract a sample of the tissue or of a fluid from the biological tissue through the needle and into the second cavity. Here, the second cavity may provide a storage compartment for biological tissue or a biological fluid extracted by means of a biopsy procedure.

When the first cavity is filled or provided with a narcotic agent narcotizing and delivery of a further medicament initially provided in the second cavity requires piercing of the skin of a patient only once. Delivery of the narcotic agent and delivery of a subsequent pharmaceutic liquid substance can be conducted during and with a single injection procedure. The skin of the patient only has to be punctured once. Moreover, with the presently proposed container it is also inherently guaranteed, that the first injectable medicament and the second injectable medicament will be delivered at the same position or region inside the punctured area of the skin or biological tissue.

According to another example the first injectable medicament and the second injectable medicament comprise different drug formulations. For instance, the first injectable medicament comprises a narcotic active ingredient whereas the second injectable medicament comprises a vaccine, a chemotherapeutic agent or some other type of pharmaceutic drug formulation as will be mentioned below. When the first and the second injectable medicaments comprise different drug formulations a combined therapy can be provided when using the container. A user or healthcare giver does no longer have to select or to prepare the different drug formulations that are intended for a combined therapy. The at least two injectable medicaments are prefilled inside the container at a given and predefined ratio. The amount and/or the ratio of the first and the second injectable medicaments do not have to be manually adjusted by the end user. Insofar the process of selecting and subsequently injecting the at least first and second injectable medicaments can be simplified. At the end, patient safely can be enhanced.

The container is not limited to the subsequent injection of only a first and a second injectable medicament. It is even conceivable, that the container does not only comprise a single membrane but that the container comprises two or even more membranes further separating the hollow space or the interior of the elongated body into three, four or even more cavities. In this way the container may even be configured to hold at least three injectable medicaments inside a respective number of hermetically separated cavities. Upon and during delivery the cavities and the membranes separating the cavities will be displaced towards the outlet until they become pierced by the needle so as to establish a fluid communication between the needle and the number of cavities in a sequential order.

According to another example the viscosity of the second injectable medicament is comparatively large. It is larger than 1.5 mPa s, larger than 3.0 mPa s. The viscosity of the second injectable medicament may be even larger than 6.0 mPa s, larger than 12 mPa s, larger than 20.0 mPa s or larger than 40 mPa s when measured at room temperature (20° C.). Depending on the size, e.g. the diameter of the hollow needle, comparatively large dispensing forces or a comparatively large pressure has to be applied to the piston in order to urge the highly viscose second injectable medicament through the hollow needle. Hence, for expelling of the at least second injectable medicament through the needle a comparatively large dispensing pressure has to be applied to the piston. A highly viscose second injectable medicament may be suitable for use with a membrane that is deformable along the longitudinal axis rather than being slidably displaceable inside the body of the container.

Depending on the flexibility or elasticity of the membrane, with a highly viscose second injectable medicament the distally directed deformation of the membrane may persist and may remain, even when the membrane is punctured or pierced by the needle. The comparatively large pressure on the piston in distal direction that is required to expel the second injectable medicament through the needle might be this large, that the membrane when punctured by the needle substantially remains in the deformed configuration. Otherwise and if the viscosity of the second injectable medicament would be rather moderate or low, e.g. in a region of 1.0 mPa s the membrane may be subject to an elastic relaxation as it is punctured by the needle, since the at least second medicament contained in the second cavity will flow through the needle.

When pierced by the needle the second injectable medicament may flow through the needle thereby immediately lowering the fluid pressure inside the second cavity. As a consequence the portion of the membrane displaced towards the distal direction and pierced by the needle may be subject to a displacement or movement towards the proximal end of the body while the piston remains stationary inside the body. Then, the fluid communication between the second cavity and the needle may be abrogated and a further displacement of the piston towards the distal direction will lead to a repeated expelling of the first injectable medicament provided in the first cavity until the membrane is again deformed or displaced to such an extent that it is pierced by the needle which is kept stationary inside the needle receiving subsection of the body.

In a further example the container comprises a cartridge configured for insertion into an injection device or into a drug delivery device. The outlet of the container may comprise a seal pierceable by the needle. In particular, the outlet may comprise a sealing disc or a rubber seal to be pierced and penetrated by the needle. The needle may be attachable to the container and/or to the cartridge. A fluid communication between the needle and the interior of the container may be also established by placing the container inside an injection device and by attaching the needle to an outlet end of the injection device. The container may comprise a standard size of a cartridge. In this way an injection device originally designed and configured for injecting of a medicament from a cartridge can be used for expelling the first injectable medicament and the second injectable medicament from the container. Moreover, also a large variety of injection needles may be available for connection with the container.

The container may be prefilled with the first liquid injectable medicament and with the second liquid injectable medicament provided in the first cavity and in the second cavity, respectively. After use, the container together with the injection device may be intended to become discarded.

According to another aspect an injection device for administering at least a first injectable medicament is provided. The injection device comprises a container as described above and a needle attached to the outlet of the container. The needle comprises a proximal tipped end extending into the needle receiving subsection. The needle may be detachably or undetachably connectable to the outlet of the container. The needle may be permanently attached to the outlet of the container or may even form the outlet of the container. Typically, the elongated body comprises a vitreous material, such as glass that is inert to the first and/or to the second injectable medicaments.

In one example the needle is immovably fixable to the outlet of the container. In other examples the needle is immovably fixed to the outlet of the container. When arranged or fixed to the outlet of the container a proximal end of the needle, which may be a tipped end, is located inside the first cavity while an opposite end of the needle, i.e. the distal end thereof is located outside the container. When arranged, attached or fixed to the outlet of the container the needle may penetrate or reach through the outlet.

The container can be intended for a single use and for a single injection procedure. Then, the prefilled container is typically intended to become discarded after use. The injection device may be also of disposable type. The container may be readily assembled inside the injection device.

Alternatively, the injection device and/or the container is of reusable type. It may be configured to accommodate the container for conducting a dispensing procedure. After the termination of the dispensing procedure the injection device may be disassembled in order to extract the container therefrom and to receive a new container for a subsequent dispensing procedure. With a reusable injection device the needle is typically attachable to an outlet end of the injection device thereby establishing a fluid communication with the outlet of the container located inside the injection device. With a disposable type of injection device the needle may be prefixed and may be permanently fixed to the outlet of the container. The needle may be molded into the body of the container. It may intersect the outlet or may even form the outlet of the container. The injection device may comprise a syringe substantially consisting of the container with a needle attached to the outlet of the container and with a plunger to be attached or being attachable to the piston of the container. The container, in particular the elongated body thereof may comprise all may be provided with radially outwardly extending finger lobes or finger flanges that allow and support a manual injection procedure.

According to another example the injection device further comprises a plunger configured to exert distally directed pressure to the piston. The plunger is at least configured to exert a driving pressure or a driving force onto the piston in distal direction. In this way the piston can be displaced and moved towards the distal end of the body of the container. The plunger may comprise an elongated rod configured to directly abut with a proximal thrust receiving surface of the piston. The plunger may be also provided with a radially widened pressure piece at its distal end. Here, the pressure piece is configured to get in direct engagement or abutment with the proximal side of the piston. The radially widened pressure piece serves to exert a rather homogeneous pressure to the piston for driving the same in distal direction. At a proximal end the plunger may comprise a radially widened abutment face, e.g. for a thumb of a user. In combination with radially outwardly extending finger lobes finger flanges connected or attached to the proximal end of the container the injection device resembles a syringe that is configured configured to conduct a manually operated expelling of the at least first and second injectable medicaments.

Typically, the elongated body is translucent so as to enable visual inspection of the content of the container. In this way the first and the second injectable medicaments contained inside the first and second cavities can be visually inspected. Moreover, even after long-term storage it may be visually inspected whether the membrane separating the first and the second cavities remains intact. It may be visually controlled, if and in how far the first and second cavities are sufficiently hermetically separated from each other, especially when the first and second medicaments are of different color or comprise different light absorption capabilities. In addition, a translucent or transparent body provides visual inspection and surveillance of the injection process. A care giver or the patient himself is enabled to visually inspect that moment during the injection procedure when the membrane is punctured by the needle.

According to another example the plunger is rigidly connected to the piston to transfer a distally directed and a proximally directed driving force from the plunger to the piston. A rigid connection may be established by, e.g. a threaded connection between the plunger and the piston. For this, the piston may comprise a threaded receptacle to receive a threaded portion at the distal end of the plunger. It is even conceivable, that the plunger is frictionally engaged with the piston or that the plunger is adhesively attached to the piston. A rigid connection between the plunger and the piston enables a withdrawal of the piston in proximal direction by means of the plunger. In this way a liquid substance or tissue may be withdrawn from an injection site of a patient after the membrane has been punctured by the needle. By displacing the piston in proximal direction a bodily fluid or biological tissue can be sucked into the second cavity. With a rather inflexible membrane the fluid communication between the second cavity and the needle remains intact once the membrane has been pierced by the needle. Withdrawal of the piston in proximal direction then has no substantial influence on the position of the membrane.

In a further example the needle is displaceable relative to the body of the container along the longitudinal axis. In particular, the needle is displaceable between at least two longitudinal positions with regard to the body. In both of the at least two longitudinal positions the needle is fixable relative to the body. In this way, the penetration depth of the needle into the hollow space of the elongated body can be varied. The depth of needle penetration determines the axial elongation of the needle receiving subsection of the body. The penetration depth or the longitudinal or axial position of the needle relative to the body determines the longitudinal or axial position of the membrane at which the membrane becomes pierced by the needle.

The longitudinal position of the proximal end of the tipped needle finally determines the amount of the first injectable medicament to be dispensed through the needle. If the proximal end of the needle extending into the needle receiving subsection is located close to the outlet, almost the entirety of the first injectable medicament contained in the first cavity will be expelled before the membrane is punctured or penetrated by the needle. In other configurations, wherein the proximal end of the needle is located at a predefined and comparatively large distance to the outlet the membrane will be pierced comparatively early, i.e. before the first injectable medicament has been completely dispensed.

As the membrane is pierced expelling of the second injectable medicament commences and a residual portion of the first injectable medicament may remain inside the container. In effect, by modifying and varying the axial position or longitudinal position of the proximal end of the needle relative to the container and hence relative to the membrane the total amount of the first injectable medicament to be dispensed from the container can be varied accordingly. Longitudinal displacement of the needle may be obtained by making use of differently sized spacers or by making use of differently configured needle assemblies configured for attachment to an interface of the injection device, wherein differently configured needle assemblies comprise double tipped injection needles of different length at least with regard to their proximal extension. Insofar, the injection device may be equipped and provided with a variety of differently configured needles each having a common interface for attachment to the outlet of the container but having double tipped injection needles with different proximal length.

According to a further example the injection device comprises a housing to accommodate the container and further comprises a drive mechanism configured to urge the plunger towards the distal end. The injection device may comprise a pen type injector configured for a single injection procedure. The housing may comprise a cartridge holder section to receive the container therein. The cartridge holder section may further comprise an open distal end provided with a needle mount. The needle mount may comprise a threaded socket to receive a correspondingly threaded section of a needle assembly, e.g. in form of a needle hub such that the proximal end of the double tipped injection needle penetrates the outlet of the container, e.g. a septum of a cartridge in order to gain access to the first cavity of the container upon assembly of the needle assembly to the housing of the injection device. The drive mechanism may comprise a piston rod configured to exert distally directed pressure to the piston of the container. The drive mechanism typically comprises at least a dose button configured to trigger and/or to control the forward movement of the piston relative to the body of the container.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while p and E have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present invention without departing from the spirit and scope of the invention. Further, it is to be noted, that any reference numerals used in the appended claims are not to be construed as limiting the scope of the invention.

### BRIEF DESCRIPTION OF THE DRAWINGS

In the following, numerous examples of the container and of an injection device will be described in greater detail by making reference to the drawings, in which:

FIG. 1 shows an example of an injection device,

FIG. 2 shows the injection device partially disassembled,

FIG. 3 shows a distal end of the injection device with a needle assembly attached to an outlet end of the injection device,

FIG. 4 is a longitudinal cross-section through an example of the container in an initial configuration,

FIG. 5 represents the container according to FIG. 4 with a needle attached to the outlet,

FIG. 6 shows the container according to FIG. 5 when the membrane has been pierced by the needle and when delivery of the second injectable medicament takes place,

FIG. 7 shows a position of the needle that varies from the position of the needle as shown in FIG. 6,

FIG. 8 is an example of the container after the membrane has been pierced by the needle,

FIG. 9 shows a configuration of the container according to FIG. 8 after or during withdrawal of the piston in proximal direction,

FIG. 10 shows a further example of a container with a deformable membrane in an initial configuration and

FIG. 11 shows the container according to FIG. 10 in a configuration in which a portion of the deformed membrane is pierced by the needle.

### DETAILED DESCRIPTION

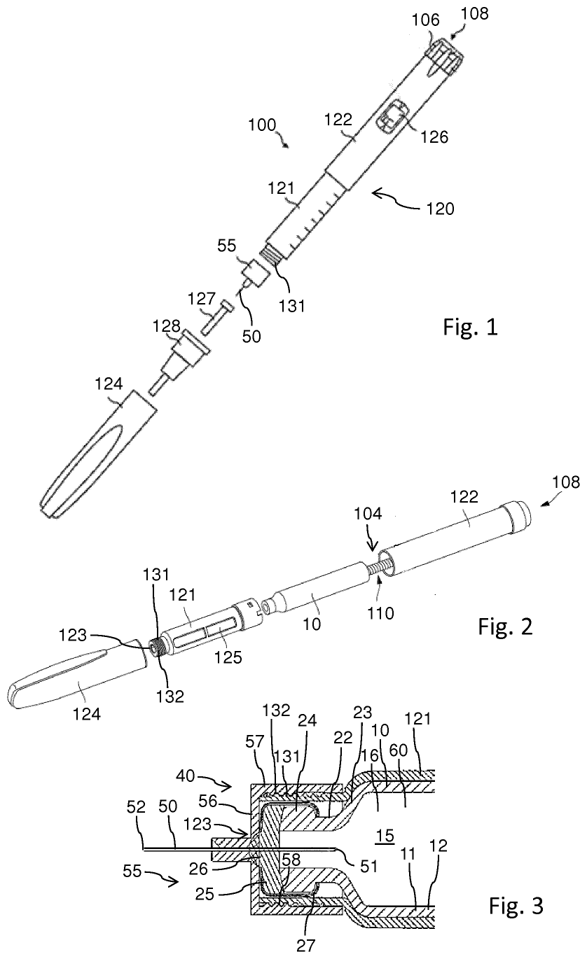

In FIGS. 1-3 an example of an injection device 100 configured as a pen type injector is illustrated. The injection device 100 comprises a housing 120. The housing 120 comprises a cartridge holder 121 and a body 122. The cartridge holder 121 is configured to accommodate a container 10 that may comprise a cartridge that is prefilled with at least a first injectable medicament 60. The cartridge holder 121 and the body 122 may be permanently or releasably attached to each other. The injection device 100 may be configured as a disposable injection device with the container 10 readily assembled therein. Alternatively, the injection device 100 may be configured as a reusable device. Here, the cartridge holder 121 can be disconnected from the body 122 in order to replace or to exchange a container 10.

The cartridge holder 121 as illustrated in FIG. 2 comprises a window 125 in order to allow visual inspection of the container 10 located therein. Near a distal end the cartridge holder 121 comprises a socket 131 having an outer threaded section 132. The socket 131 is configured to engage with a needle assembly 55 which is separately illustrated in FIG. 1. The needle assembly 55 comprises an injection needle 50. The injection needle 50 is a double tipped hollow cannula having a proximal end 51 and a distal end 52 as shown in FIG. 3. The needle assembly 55 also denoted as a needle hub comprises a bottom section 56 and a sidewall section 57. The bottom section 56 and the sidewall section 57 form a cup-shaped receptacle configured to receive the threaded socket 131 of the cartridge holder 121. The sidewall section 57 comprises an inner threaded section 58 that mates with the outer threaded section 132 of the socket 131. A distal end face of the cartridge holder 121 comprises a through opening 123 through which the proximally protruding portion of the needle 50 can extend into the interior of the cartridge holder 121 and hence into the interior of the cartridge or container 10 when the needle assembly 55 is attached to the cartridge holder 121 and when the container 10 is arranged in the cartridge holder 121.

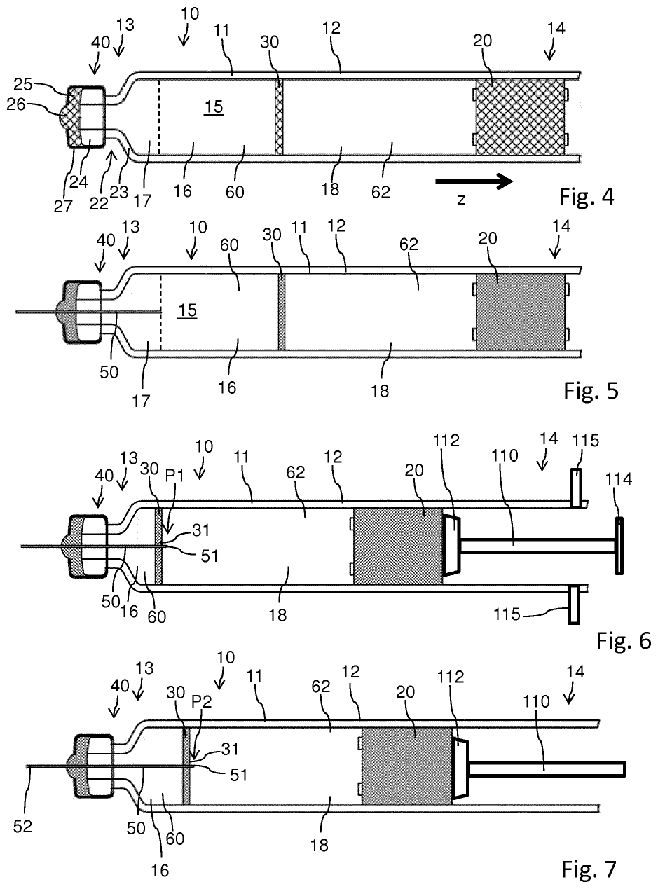

The container 10 is arranged inside the cartridge holder 121. It is positionally fixed inside the cartridge holder 121. The container 10 may comprise a narrowing shoulder portion 23 to abut or to engage axially with a correspondingly-shaped shoulder section of the cartridge holder 121. The container 10 comprises an elongated and tubular-shaped body 11. The body 11 may comprise a vitreous body. The body 11 may be made of glass. The body may be translucent or transparent in order to allow visual inspection of the content of the container 10. The elongated body 11 extends along a longitudinal direction (z). The body 11 comprises a distal end 13 and an oppositely located proximal end 14 as illustrated in FIG. 4.

With the distal end the body 11 is arranged near or at the distal end of the cartridge holder 121. The distal end 13 of the body 11 comprises a narrowing shoulder portion 23 extending into a diameter reduced neck portion 22. At the far distal end the neck portion 22 extends into a radially widening head portion 24. At the head portion 24 there is provided a seal 25, e.g. in form of a pierceable sealing disc. This seal 25 may comprise a pierceable rubber septum 26 that is fixed to the head portion 24 and hence to the distal end 13 of the body 11 by means of a crimped cap 27. The seal 25 may form an outlet 40 of the container 10 at the distal end 13 of the elongated body 11.

The injection device 100 may be further equipped with a drive mechanism 104 comprising a plunger 110 or a piston rod. The drive mechanism 104 may be further equipped with a dose button 108 by way of which a dispensing action of the injection device 100 can be triggered or controlled. Optionally, the injection device 100 and its drive mechanism 104 comprises a dose dial 106 by way of which a size of a dose to be dispensed can be individually set or by way of which the injection device 100 can be deployed or prepared for a subsequent dispensing procedure.

Optionally and as illustrated in FIG. 1 the body 122 of the housing 120 may be provided with a dose size indicating window 126. In the window 126 the size of a dose actually set can be visually displayed thus informing the user of the amount of the medicament to be dispensed during a subsequent dispensing procedure.

As further illustrated in FIG. 1 the needle assembly 55 may be provided with an inner needle cap 127 configured to cover the distal end 52 of the injection needle 50. The entire needle assembly 55 may be further covered by an outer needle cap 128. If not in use the needle assembly 55 should be detached from the distal end of the cartridge holder 121. Then the cartridge holder 121 can and should be covered by a protective cap 124. The protective cap 124 is configured to releasably engage with at least one of the cartridge holder 121 and the body 122. Prior to assembling the needle assembly 55 to the cartridge holder 121 the protective cap 124 has to be detached from the housing 120.

The interaction of the container 10 with a pen type injection device 100 as illustrated in FIGS. 1-3 is only exemplary. The general working principle of the container does not require interaction with a pen type injection device. The container 10 may be used as a manually operated syringe as will be explained below.

The container 10 in its entirety is illustrated in FIG. 4. The container 10 comprises an elongated body 11 with a tubular-shaped sidewall 12 extending along a longitudinal axis or along an axial direction (z). The container 10 and the elongated body 11 comprise a distal end 13 provided with an outlet 40 for at least a first injectable medicament 60. At an opposite longitudinal end the body 11 comprises a proximal end 14. The elongated body 11 and its tubular-shaped sidewall 12 confine a hollow space 15 of cylindrical geometry. Towards the proximal end 14 the hollow space 15 inside the elongated body 11 is sealed by a piston 20. The piston 20 comprises a diameter that corresponds and matches with the inner cross-section of the sidewall 12. The piston is sealingly engaged with the sidewall 12 and is slidable along the longitudinal axis (z) relative to the sidewall 12.

At a given axial or longitudinal distance from the piston 20 there is arranged a membrane 30 inside the elongated body. The membrane 30 is also sealingly engaged with the sidewall 12 and is at least one of slidable and deformable along the longitudinal axis (z) relative to the sidewall 12. As seen in longitudinal direction the membrane 30 is located between the piston 20 and the seal 40. The membrane 30 may be located in a longitudinal mid-section of the container 10. The piston 20 may be provided at or near a proximal end 14 of the container 10 and the outlet 40 may be provided at the distal end 13.

The membrane 30 is substantially impenetrable to liquids or is at least substantially impenetrable to pharmaceutically active ingredients of the at least first medicament 60 and/or of an at least second medicament 62. The hollow space 15 inside the body 11 is separated by the membrane 30 into a first cavity 16 and into a second cavity 18. The first cavity 16 is confined by the sidewall 12, by the outlet 40 and by the membrane 30. The second cavity 18 is confined by the sidewall 12, by the piston 20 and by the membrane 30. As seen in longitudinal direction the first and second cavities 16, 18 are located behind one another.

Adjacent to the distal end and/or adjacent to the outlet 40 the hollow space 15 of the container 10 comprises a needle receiving subsection 17. The needle receiving subsection is defined by the penetration depth of a needle 50 configured to enter the hollow space 15 from the outlet 40 in the longitudinal direction and towards the proximal end 14. The needle receiving subsection 17 is virtually indicated in FIGS. 4 and 5 by a dashed vertical line. The proximal end of the needle receiving subsection 17 coincides with the longitudinal position of a proximal end 51 of the needle 50 when arranged inside the hollow space 15 as indicated in FIG. 5.

The axial position of the needle 50 may be variable. In an initial configuration and when delivered to a healthcare professional or to a customer the container 10 may be void of a needle. The container 10 may comprise a cartridge sealed towards the distal end 13 by a pierceable seal 25. For conducting an injection procedure the outlet 40 of the container 10 is provided with a double tipped injection needle 50. In alternative examples the needle 50 may undetachably belong to the container 10 and to the body 11. It may be molded into the body 11 and the container 10. Hence, the needle 50 may be in fluid communication with the first cavity 16 when it is commercially distributed to healthcare professionals or end users.

The second cavity 18 is typically filled with the second injectable medicament 62. Starting from the configuration as shown in FIG. 5 distally directed pressure may be applied to the piston 20 in order to drive the piston 20 towards the distal end 13. Here, pressure applied to the piston 20 transfers through the second injectable medicament 62 which is substantially incompressible. The rise of a fluid pressure of the second injectable medicament 62 leads to a respective distally directed displacement or deformation of the membrane 30 towards the distal end 13. As a consequence also the pressure inside the first cavity 16 will rise thus leading to the expelling of an amount of the first injectable medicament 60 through the needle 50. If a distal end 52 of the injection needle 50 is located in biological tissue the first injectable medicament 60 will be injected in the biological tissue accordingly.

In FIG. 6 a situation is illustrated in which the piston 20 has been displaced towards the distal end 13 in unison with the membrane 30. As the membrane 30 enters the needle receiving subsection 17 a portion 31 of the membrane 30 will become pierced and penetrated by the proximal end 51 of the needle 50. Consequently the needle 50 gets in fluid communication with the second cavity 18 and the second injectable medicament 62 provided in the second cavity 18 will become subject to expelling through the needle 50 as the piston 20 is displaced further to the distal end 13. In FIG. 6 also a plunger 110 with a radially widened pressure piece 112 is illustrated by way of which distally directed pressure is exertable onto the piston 20. The plunger 110 may belong to the drive mechanism 104 of an injection device. Alternatively and when the container is implemented as a manually operable syringe the plunger 110 may be manually depressed by a user, e.g. by a thumb of a user. For this the plunger 110 may comprise a radially widening flange 114 at a proximal end onto which a user may apply pressure with a thumb.

As further illustrated in FIG. 6 a proximal end of the body 11 may be provided with radially outwardly extending lobes 115. There may be provided two diametrically oppositely located lobes 115 or radially outwardly extending flange portion a proximal end section of the body 11. The lobes 115 may provide finger flanges, e.g. for a middle and an index finger of a user in order to apply a counterforce to the container 10 as a user exerts distally directed pressure onto the plunger 110 with a thumb. The lobes 115 may be integrally formed with the container 10. In this case the container 10 and its elongated body 11 may comprise or may be manufactured from an injection molded plastic material. Alternatively, the lobes 115 may be provided as separate components that are fixed to the sidewall 12 of the body. In this case the body 11 may be made of glass.

In the illustration of FIG. 6 the proximal end 51 of the needle 50 is located at a first position P1. The axial or longitudinal position of the proximal end 51 of the needle 50 determines the axial position at which the membrane 50 is pierced and intersected by the needle 50. As illustrated in FIG. 6 the first cavity 16 comprises a residual non-zero volume as the membrane 30 is pierced by the needle 50. A certain residual amount of the first medicament 60 is hence left inside the container 10 as dispensing and expelling of the second medicament 62 starts. Compared to FIG. 6 the needle 50 and its proximal end 51 are located at a longitudinal position P2 as illustrated in FIG. 7. Here the proximal end 51 of the needle 50 is even further away from the distal end 13 of the body 11 compared to the configuration of FIG. 6.

As a consequence, the remaining volume of the first cavity 16 is larger in FIG. 7 than in FIG. 6. Insofar, the longitudinal position of the needle 50, in particular of its proximal tipped end 51 governs and determines the total amount of the first injectable medicament to be expelled from the container 10. With a minimum distance of the proximal end 51 of the needle 50 and the outlet 40 or distal end 13 of the body 11 a maximum amount of the first injectable medicament 60 will be expelled through the needle 50. By shortening the distance between the proximal end 51 of the needle 50 and the membrane 30 in an initial configuration the smaller will be the amount of the first injectable medicament expelled through the needle 50. Consequently, with an increasing penetration depth of the needle 50 in longitudinal direction into the hollow space 15 of the container 10 the larger will be the residual amount of the first medicament 60 that remains inside the container 10.

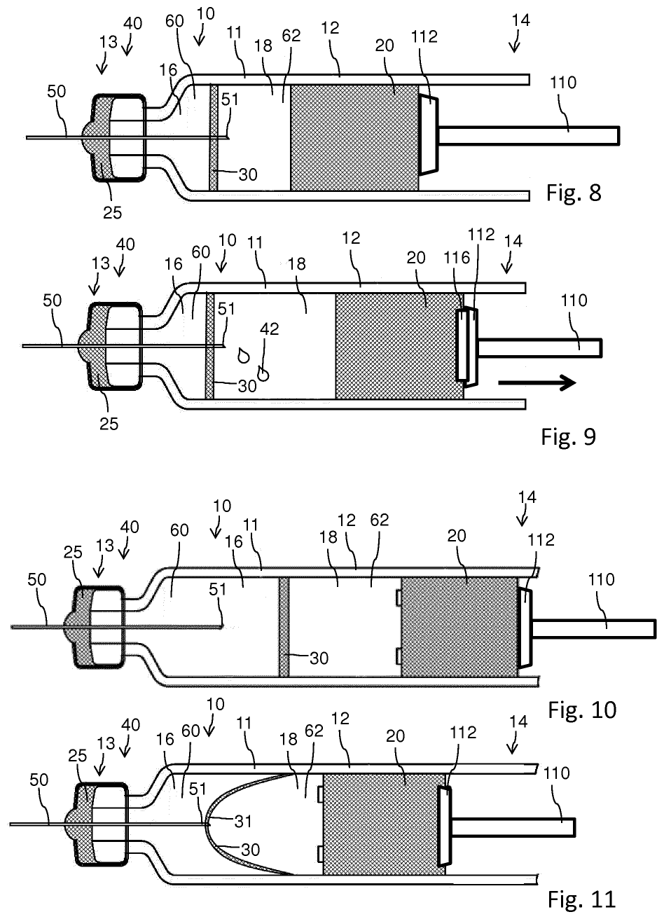

As the dispensing procedure continues the second injectable medicament 62 is urged and expelled through the injection needle 50 until a configuration as exemplary illustrated in FIG. 8 may be reached. In this configuration also a substantial amount of the second injectable medicament 62 has been expelled through the needle 50. The piston 20 may be even displaced further in distal direction until it is also pierced by the proximal tip 51 of the needle 60. Engagement of the piston 20 with the needle 50 will provide a haptic feedback to a user, that the entirety or that at least a major portion of the second injectable medicament 62 has been expelled and that the injection procedure has terminated.

The container as illustrated in FIGS. 4-8 is configured for the sequential expelling of two different injectable medicaments 60, 62 at or in the same injection site.

Furthermore and according to another aspect the container 10 may be also used for biopsy procedures. It may be then only prefilled with the first injectable medicament 60 located inside the first cavity 16. The second cavity 18 may be substantially empty and may be void of the second injectable medicament 62. For the purpose of conducting a biopsy operation there may be provided a gas between the piston 20 and the membrane 30, which under compression is configured to displace or to deform the membrane 30 towards the distal end 13 in order to expel the first injectable medicament 60 as described above in connection with FIGS. 4-7. Instead of a gas there may be provided a spacer inside the second cavity 18. The spacer may separate the piston 20 and membrane 30 in axial direction. It may be sandwiched between the piston 20 and the membrane 30. The spacer, a gas or an inert liquid substance located between the membrane 30 and the piston 20 serves to prevent that the piston 20 is pierced by the needle 50 when the membrane 30 is penetrated by the needle as the membrane 30 and the piston 20 are subject to a longitudinal displacement towards the distal end 13.

Once the membrane 30 has been penetrated by the proximal end 51 of the needle 50 the piston 20 can be withdrawn and can be displaced or moved towards the proximal end 14 of the body 11 as illustrated in FIG. 9. When withdrawing the piston 20 towards the proximal end 14 a suction effect is generated and a negative pressure is provided in the second cavity 18 thus leading to a withdrawal of tissue and/or of a biological fluid from the piercing site of a patient into the second cavity. The withdrawing and suction of a sample fluid 42 into the second cavity 18 is schematically illustrated in FIG. 9. The plunger 110 and/or its pressure piece 112 is rigidly connected to the piston 20. Here, a connection between the plunger 110 and the piston 20 may comprise a threaded engagement, a magnetic connection, a positive engagement established by snap features or an adhesive connection. Moreover, it is conceivable, that the piston 20 and the elongated plunger 110 are integrally formed.

In FIGS. 10 and 11 a further configuration of a membrane 30 is illustrated. The container 10 as shown in FIG. 10 in an initial configuration is substantially identical to the container 10 as illustrated and explained with regard to FIGS. 4 and 5. But here and contrary to the container 10 of FIG. 4 the container 10 as illustrated in FIG. 10 comprises a deformable membrane 30. In an initial configuration the proximal end 51 of the needle 50 extends into the first cavity 16. As a distally directed pressure is applied to the piston 20 the fluid pressure inside the second cavity 18 rises which leads to a distally directed deformation and bulging of the membrane 30 as illustrated in FIG. 11.

The membrane 30 may comprise an elastically deformable material, such as one of several layers of a thermoplastic elastomer. The membrane 30 and its outer circumference may be in frictional engagement with the inside of the sidewall 12. A friction force between the membrane 30 and the sidewall 12 may be substantially larger than a force or pressure required to induce a distally directed deformation of the membrane 30. As the membrane 30 deforms towards the distal end 13 the volume of the first cavity 16 substantially decreases while the volume of the second cavity remains substantially constant. Here, a portion 51, e.g. a radial central portion of the membrane 30 may reach the needle receiving subsection 17 first and may be punctured and penetrated by the proximal tip end 51 of the needle 50 as illustrated in FIG. 11.

As soon as the portion 31 of the membrane 30 is pierced a fluid transferring connection is established between the hollow interior of the needle 50 and the second cavity 18. As the second injectable medicament 62 flows through the needle 50 the fluid pressure inside the second cavity 18 may decrease thus leading to a relaxation of the membrane 30 such that the pierced portion 31 moves in proximal direction and disengages from the needle 50. A further displacement of the piston 20 in distal direction may then repeatedly lead to an expelling of a certain amount of the first injectable medicament 60 from the first cavity 16 until the portion 31 of the membrane 30 will be pierced again. Since the first and the second injectable medicaments 60, 62 will be subject to a mixing in the pierced tissue temporal variations of the order of expelling of the first and the second injectable medicaments 60, 62 might be tolerable.

Moreover, if the second medicament 62 comprises a comparatively large viscosity and if the diameter of the needle 50 is comparatively small a comparatively high pressure has to be applied to the piston 20 in distal direction. This pressure may be high enough to keep the membrane 30 in the bulged or deformed configuration as illustrated in FIG. 11. Hence, with a highly viscose second injectable medicament 62 the fluid communication between the second cavity 18 and the needle 50 may persist even after the membrane 30 has been pierced and intersected by the proximal end 51 of the needle 50.

In some examples or embodiments the membrane 30 might be substantially inflexible. It may exhibit a well-defined sliding behavior with regard to the tubular-shaped sidewall 12. In other embodiments the membrane 30 may be in frictional engagement with the sidewall 12 to such an extent that an elastic membrane 30 tends to deform or to bulge towards the distal end 13 as a respective fluid pressure is applied to the membrane 30. The membrane 30 may be flexible as well as stretchable in order to become pierced by the needle 50 as it is deformed towards the distal end 13.

Depending on the elastic properties of the material the membrane 30 is made of and depending on the overall geometry, e.g. depending on the thickness of the membrane in longitudinal direction and its diameter or cross-section the membrane 30 may be also subject to a combined sliding movement relative to the sidewall 12 as well as to a bulging or deformation due to a comparatively high pressure in the second cavity 18.

### LIST OF REFERENCE NUMBERS

10 container
11 elongated body
12 sidewall
13 distal end
14 proximal end
15 hollow space
16 first cavity
17 needle receiving subsection
18 second cavity
20 piston
22 neck portion
23 shoulder portion
24 head portion
25 seal
26 septum
27 cap
30 membrane
31 portion
40 outlet
42 sample fluid
50 needle
51 proximal end
52 distal end
55 needle assembly
56 bottom section
57 sidewall section
58 threaded section
60 medicament
62 medicament
100 injection device
104 drive mechanism
106 dose dial
108 dose button
110 plunger
112 pressure piece
114 flange
115 lobe
116 connection
120 housing
121 cartridge holder
122 body
123 through opening
124 cap
125 window
126 window
127 inner needle cap
128 outer needle cap
131 socket
132 thread

1. A container (10) for at least a first injectable medicament (60), the container comprising:
an elongated body (11) having a tubular-shaped sidewall (12) extending along a longitudinal axis (z) and having a distal end (13), wherein the elongated body (11) comprises a hollow space (15) with a needle receiving subsection (17) adjacent to the distal end (13),
an outlet (40) at the distal end (13) of the body (11),
a piston (20) arranged inside the elongated body (11), sealingly engaged with the sidewall (12) and slidable along the longitudinal axis (z) relative to the sidewall (12),
a membrane (30) arranged inside the elongated body (11) sealingly engaged with the sidewall (12) and being at least one of slidable and deformable along the longitudinal axis (z) relative to the sidewall (12),
a first cavity (16) confined by the sidewall (12), the outlet (40) and the membrane (30), wherein the first injectable medicament (60) is located in the first cavity (16),
a second cavity (18) confined by the sidewall (12), the piston (20) and the membrane (30), wherein the piston (20) and the membrane (30) are separated from each other by a distance along the longitudinal axis (z),
wherein the outlet (40) is configured to receive or to hold a needle (50) extending from the distal end (13) into the needle receiving subsection (17), and
wherein in an initial configuration, the membrane (30) is located outside the needle receiving subsection (17) and wherein the membrane (30) is displaceable or deformable relative to the sidewall (12) to such an extent that at least a portion (31) of the membrane (30) enters the needle receiving subsection (17).

2. The container (10) according to claim 1, wherein the membrane (30) is pierceable and penetrable by the needle (50).

3. The container (10) according to any one of the preceding claims, wherein at least a second injectable medicament (62) is located in the second cavity (18).

4. The container according to any one of the preceding claims, wherein the membrane (30) is substantially inflexible.

5. The container according to any one of the preceding claims, wherein the membrane (30) comprises at least one or a combination of the following materials: a thermoplastic polymer, a thermoplastic elastomer, a thermoplastic vulcanizate, polyethylene (PE), polyvinyl chloride (PVC), polytetrafluoro-ethylene (PTFE), silicone rubber, halogenated butyl rubber, chlorobutyl rubber, bromobutyl rubber, chemical vapor deposited poly(p-xylylene) polymers, parylene, cork.

6. The container (10) according to any one of the preceding claims, wherein the membrane (30) comprises a thickness in a range from 10 μm-2 mm, from 10 μm-100 μm, from 100 μm-2 mm, or from 100 μm-1 mm.

7. The container (10) according to any one of the preceding claims, wherein the membrane (30) is substantially impenetrable to liquids.

8. The container (10) according to any one of the preceding claims 3 to 7, wherein the at least first injectable medicament (60) comprises a narcotic active ingredient.

9. The container (10) according to any one of the preceding claims 3 to 8, wherein the first injectable medicament (60) and the second injectable medicament (62) comprise different drug formulations.

10. The container (10) according to any one of the preceding claims 3 to 9, wherein a viscosity of the second injectable medicament (62) is larger than 1.5 mPa s, larger than 3.0 mPa s, larger than 6.0 mPa s, larger than 12.0 mPa s, larger than 20.0 mPa s, or larger than 40 mPa s when measured at room temperature (20° C.).

11. The container (10) according to any one of the preceding claims, wherein the outlet (40) comprises a seal (25) pierceable by the needle (50).

12. An injection device (100) for administering at least a first injectable medicament (60), the injection device (100) comprising a container according to any one of the preceding claims and a needle (50) attached to the outlet (40) of the container (10), wherein the needle (50) comprises a proximal tipped end (51) extending into the needle receiving subsection (17).

13. The injection device according to claim 12, further comprising a plunger (110) configured to exert distally directed pressure to the piston (20).

14. The injection device according to claim 13, wherein the plunger (110) is rigidly connected to the piston (20) to transfer a distally directed and a proximally directed driving force from the plunger (110) to the piston (20).

15. The injection device according to any one of the preceding claims 11 to 14, wherein the needle (50) is displaceable relative to the body (11) of the container (10) along the longitudinal axis (z) between at least two longitudinal positions and wherein the needle (50) is fixable relative to the body (11) in any of the at least two longitudinal positions.

16. The injection device according to any one of the preceding claims 11 to 15, further comprising a housing (120) to accommodate the container (10) and comprising a drive mechanism (104) configured to urge the plunger (110) towards the distal end (13).
